(19) United States
(12) Patent Application Publication
(10) Pub. No.: US 2003/0026771 A1
(43) Pub. Date: Feb. 6, 2003

(54) Thixotropic base for dispersion of dimethicone coated inorganic materials

(76) Inventor: Robert B. Hopp, Richland, WA (US)

Correspondence Address: STEPHEN R. MAY, 412 BROADMOOR ST., RICHLAND, WA, 99352, US

(21) Appl. No.: 09/919,674

(22) Filed: Jul. 31, 2001

(51) Int. Cl. A61K 7/42; A61K 7/44; A61K 7/00

(52) U.S. Cl. 424/59; 424/60; 424/400; 424/401

(57) ABSTRACT

A well-dispersed thixotropic sunscreen composition having no greasy or powdery after effects is provided, along with a method for their production. The composition comprises an oleaginous liquid base, a thickening agent, and a coated particulate sunscreen material. The sunscreen agents are coated with, for example, dimethicone, which enables van der Waals forces to provide hydrogen bonding, keeping the sunscreen agents in suspension and without appreciable reaggregation.

### FIELD OF THE INVENTION

The present invention relates to the dispersion of coated inorganic particulate materials in a thixotropic base.

### BACKGROUND OF THE INVENTION AND PRIOR ART

As health concerns mount about exposure to the harmful effects of the ultraviolet (UV) components of the sun, cancer-conscious consumers are becoming more diligent about the application of sunscreen to exposed skin, such as facial areas, ears and arms and legs. The particle size of inorganic sunscreen molecules such as zinc oxide and titanium dioxide can have a significant effect on the resulting sun protection factor (SPF) of the sunscreen. If an extremely fine particulate size can be maintained in suspension in the sunscreen the SPF increases per unit of inorganic sunscreen. The ideal particle size of, for example, zinc oxide, in a sunscreen is in the range of 80 to 150 nm; this size best attenuates UV and minimizes the scattering of visible wavelengths and reduces the cosmetic lightening effect associated with these particles. Particulate Sun Blocks: General Principles. Fairhurst, D and Mitchnick, M. Sunscreen Development, Evaluation and Regulatory Aspects, 1997, New York, N.Y., pg 324.

Fine particulates are produced by the process of deagglomeration, and dispersion of fine inorganic particulates into solution is the first step in obtaining a uniform distribution of small particulates in a sunscreen. This involves the wetting of the particulates where the solvent contacts the inorganic particulates. Lyophilic surfaces are easily wetted without the need for surfactants. The particulates are stabilized in solution so that they do not reaggregate, which is critical for long-term stability (and shelf life) of the sunscreen products.

In the context of the present invention, thixotropic mixtures are anhydrous compositions of lipophilic molecules primarily composed of carbon and hydrogen. These mixtures are made by the rapid chilling of the two components through a cloud point that sets up a semi-solid gel matrix. This gel matrix is formed and maintained by intramolecular hydrogen bonding between carbon and hydrogen, the so-called van der Waals forces.

When dimethicone coated inorganic particulates are dispersed into a thixotropic mixture above the cloud point, the particulates can be maintained in a dispersed state by rapid cooling and formation of a gel matrix. If the chilling of the solution occurs rapidly with vigorous stirring, inorganic particulates coated with dimethicone present at the time of cloud point formation, are locked into the stable gel matrix. van der Waal bonding of the methyl groups on the coated organic particles with the surrounding components of the thixotropic matrix holds the particles in the matrix in a stable and dispersed state.

Rapid formation of the gel matrix when rapidly chilled causes the solution to pass through the cloud point and produces van der Waals bonding of the dimethicone coated inorganic molecules to the surrounding solvent molecules that are present in abundance. Because of their large molecular weight, these solvent molecules also bind to other surrounding solvent molecules making up the gel matrix. After formation of the gel matrix the inorganic particles are not subject to diffusion or settling and are held securely in the base. This matrix prevents reaggregation of the molecules and allows their long-term storage and use. The stable suspended state of the inorganic particles maintains the optimal size needed for maximal sun protection and provides optimal particle size of the inorganic particle in a sun screen product.

Thixotropic compounds are well known in the art. For example, in U.S. Pat. No. 2,628,187, a thixotropic base containing mineral oil and polyethylene is produced, and uncoated zinc oxide powder is dispersed therein, with a colloid or pigment mill after cooling of the polyethylene and mineral oil base. Similarly, U.S. Pat. No. 3,215,599 discloses addition of uncoated zinc oxide to a mineral oil-polyethylene base. The zinc oxide is added after the base has passed through the cloud point.

U.S. Pat. No. 3,733,403 discloses the use of magnesium silicate as an agent to prevent synuresis, and U.S. Pat. No. 4,083,956 discloses the production of an anhydrous antiperspirant composition in the form of a thixotropic cream. A non-greasy composition useful as a cosmetic base is disclosed in U.S. Pat. No. 4,164,563, by heating a liquid base material (mineral oil, wax or paraffin) and adding a solid agent such as cetyl alcohol, and later addition of inorganic powders such as hydrated magnesium and aluminum silicates.

Further, U.S. Pat. No. 4,310,516 discloses an anhydrous pseudoplastic thixotropic base, by mixing a solid emulsifying agent and a liquid oleaginous material. U.S. Pat. No. 5,914,101 describes dispersion of silica-coated zinc oxide particles in water with stabilizing agents to prevent reaggregation of the zinc oxide in the water base, while U.S. Pat. No. 5,939,054 discloses wetted titanium dioxide or zinc oxide particles incorporated into an emulsified water-based sunscreen.

Finally, U.S. Pat. No. 6,165,450 discloses an oil-in-water suspoemulsion sunscreen formulation to deliver inorganic sunscreens such as titanium oxide and zinc oxide as a sprayable composition.

### OBJECTS OF THE INVENTION

It is an object of the present invention to prepare a thixotropic oleaginous sunscreen product that has an effective amount of organic or inorganic sunscreening agents to provide sufficient SPF protection.

It is a further object to provide a sunscreen product that has excellent “rub-in” properties wherein the sunscreening agents remain suspended in the product over a wide temperature range of expected use.

### SUMMARY OF THE INVENTION

The present invention is a method of preparing a sunscreen composition, and the sunscreen prepared thereby. The method comprises the steps of: preparing a thixotropic base material by combining (on a weight basis) from 25-90% oleaginous liquid; 5-35% thickening agent and 0.5-25% of coated particulate material having sun blocking properties, wherein the oleaginous liquid and thickening agent are mixed and heated to a temperature of greater than 80° C., then mixing in a second portion of the oleaginous liquid at a temperature less than 8° C., and finally mixing the components until it cools sufficiently for packaging.

More particularly, the method comprises the steps of rapidly mixing a first mixture of a thickening agent and the oleaginous liquid at a temperature greater than 100° C.; heating a second mixture of the oleaginous liquid and the sunscreen material to a temperature greater than 100° C.; combining the two mixtures; cooling a portion of the oleaginous liquid to a temperature less than 8° C. and adding it to the combined first mixture.

The sunscreen material produced by the method of the present invention comprises: an oleaginous liquid base in an amount of from 25-90% by weight; a thickening agent in an amount of from 5-35% by weight, and a coated particulate sunscreen material in an amount of from 0.5-25% by weight. Preferably, the oleaginous liquid base comprises isopropyl myristate; the thickening agent comprises glycerol monosterate; and the coated sunscreen material comprises zinc oxide coated with dimethicone.

### DETAILED DESCRIPTION OF THE INVENTION

The compositions of the present invention comprise novel sunscreen compositions having excellent skin coverage, without a greasy feel or powdery film, and with excellent SPF protection. The compositions are stable at both the high and low end of the temperature ranges expected to be encountered by any one wearing the compositions. The compositions are thixotropic—that is, while they are colloidal suspensions during manufacture, they remain in their stable gel form through packaging, storage, application and use by the consumer.

Sunscreen compositions made according to the present invention may be prepared from a number of different compositions. For example, the liquid oleaginous base for each are preferably liquid hydrocarbons such as fatty alcohols, vegetable oils, mineral oils, white petrolatum, lanolin alcohols and lanolin. Representative synthetic oils that may be used herein include, but are not limited to, isopropyl myristate, oleyl myristate, methyl isopropyl and butyl esters of fatty acids and acetoglyceride esters. The preferred base material is isopropyl myristate. Preferably, the oleaginous base is present in an amount of from 25-90%, by weight.

Because the products of the present invention may be marketed as either creams or ointments, thickening agents are typically added. Representative thickening agents include polyethylene, polypropylene, polybutylene, polymethylbutylene copolymer, and copolymers of olefins and acetates such as ethylene-vinyl acetate copolymer. Sorbitan fatty esters such as sorbitan monolaurate and oleate, beeswax, paraffin, and paraffinic waxes can also be used. Useful aliphatic monomeric compounds include cetyl alcohol, stearic acid, cholesterol, glycerol monosterate, and glycerol disterate. The preferred thickening agent is glycerol monosterate. Preferably, the thickening agent is present in an amount of from 5-35% by weight.

Sun screening agents are well known in the art, and are preferably inorganic compounds such as dimethicone coated zinc oxide, dimethicone coated titanium dioxide and dimethicone coated iron oxides. Organic sunscreen agents can be added to the inorganic agents in order to enhance the SPF of the product. For example, organic agents may include aminobenzoic acid, cinoxate, diethanolamine methoxycinnamate, digalloyl trioleate, dioxybenzone, ethyl-4-[bis(hydroxypropyl)] aminobenzoate, octyl methoxycinnamate, octyl salicylate, glyceryl aminobenozoate, homosalate, lawsone with dihydroxyacetone, menthyl anthranilate, ocyocyrlene, oxybenzone, Padimate O, phenylbenzimidazone sulfonic acid, red petrolatum, sulisobenzone and trolamine salicylate. Preferably, the sunscreen agents are present in an amount of from 0.5-25% by weight.

Both organic and inorganic sunscreen agents are commercially available from a number of different suppliers. For example, both zinc oxide and titanium dioxide used in sunscreen formulations generally has a particle size of from 5-150 nm.

Because the most likely candidates for sunscreen agents possess intramolecular dipolar energies, there is a strong tendency for reaggregation of these molecules in solution after they have been suspended during production of the sunscreen product. Uncoated zinc oxide or titanium dioxide will reaggregate in solution unless molecular forces are present that prevent the interaction of these intramolecular dipolar energies. Dimethicone coating of the inorganic sunxcreen molecules makes van der Waals forces available for hydrogen bonding of the methyl groups with the surrounding solvent. Therefore, the van der Waals forces can be used to prevent the reaggregation of inorganic sunscreen particulates.

Because these compounds are applied much as are cosmetics, the “feel” of the products must mimic as much as possible other cosmetic preparations consumers are familiar with. To that end, agents may be added to improve the “rub-in” of the compounds so that they do not leave unsightly greasy stains or powdery residues on the surface of the skin. For example, silicones (such as dimethicone 1236 from the General Electric Company), or other liquid or particulate silicones may be used either singly or in combination with one another. These compounds may be added in an amount of from 1-25%, by weight.

Multiple thickening agents may be used in combination with one another in the same composition to give maximum viscosity control in the product. Multiple liquid bases or inorganic particulates may also be used to increase the SPF of the final product.

The product of the present invention reduces reliance on the physical support of the individual zinc (or titanium) oxide particles and relies instead on chemical bonding provided by van der Waals forces. In an uncoated state the zinc oxide molecules have no attraction to the surrounding hydrophobic nonpolar base materials and would therefore likely thereafter reagglomerate and settle out of solution. However, by using dimethicone coated zinc oxide added above the cloud point, and producing a rapid chilling of the mineral oil and polyethylene thixotropic mixture, the dimethicone of the zinc oxide molecules binds to the polyethylene and mineral oil.

### EXAMPLES

A plurality of sunscreen compositions were prepared according to the present invention as set forth below in the following Examples.

### Example #1

A mixture of a thickening agent (such as glycerol monosterate (22% by weight)) and an oleaginous liquid base (such as isopropyl myristate (33% by weight)) was prepared and heated to 100° C. with slow mixing. The temperature of the mixture must be above the melting point of the thickening agent, and in the examples presented herein will be within a range of approximately 90-130° C. When the solution became clear it was removed from the heat and placed under a mixer capable of rapid dispersion mixing. A solution of isopropyl myristate (8%) containing 4% dimethicone zinc oxide was heated to a temperature of 106° C. This solution was stirred rapidly while heating. The solutions were then combined and the temperature monitored until it cooled to a temperature of approximately 2 degrees above the cloud point, or approximately 57° C. A second portion of isopropyl myristate (33% by weight) was cooled to 4° C. and then added rapidly to the 57° C. solution with vigorous stirring. The cloud point was quickly reached and the solution became opaque. The mixture was continuously mixed until it cooled sufficiently for packaging.

### Example #2

Glycerol monosterate (22% by weight) was mixed with isopropyl myristate (22% by weight) and heated to 100° C. with slow mixing to form solution #1. When the solution became clear it was removed from the heat and mixed with rapid dispersion. A portion of isopropyl myristate (10% by weight) containing 11% dimethicone coated zinc oxide was heated to a temperature of 106° C. with rapid stirring to form solution #2. The two solutions were then mixed together to form solution #3 and cooled to 57° C., at which point a fourth solution of isopropyl myristate (20% by weight) chilled to 4° C. was rapidly added to the third solution accompanied by vigorous stirring to form solution #5. The cloud point was quickly reached and solution #5 became opaque. Dimethicone (15% by weight) at room temperature was added to solution #5 and continued to cool until suitable for packaging.

### Example #3

Glycerol monosterate (22% by weight) was mixed with isopropyl myristate (22% by weight) and heated to a temperature of 11° C. with slow mixing to form solution #1. When solution #1 became clear it was removed from the heat and placed under a mixer capable of rapid dispersion mixing. Solution #2 comprising a solution of isopropyl myristate (10% by weight) with 9% dimethicone zinc oxide was heated to a temperature of 106° C. and then added to solution #2 to form solution #3. Isopropyl myristate (16.5% by weight) cooled to 4° C. was added with rapid mixing to solution #3 when solution #3 had cooled to 57° C. to form solution #4. The cloud point was quickly reached and the solution became opaque. Dimethicone (15% by weight) at room temperature, and octyl methoxycinnamate (7.5% by weight) were added to solution #4 with mixing until cooled to a suitable temperature for packaging.

### CLOSURE

From the foregoing description, one skilled in the art can easily acertain the essential characteristics of this invention and, without departing from the spirit and scope thereof, can make various changes and modifications of the invention to adapt it to various other uses. Accordingly, the scope of the present invention is to be limited solely to the scope of the claims appended hereto.

I claim:

1.A method of preparing a sunscreen composition comprising the steps of:
a. preparing a thixotropic base material by combining (by weight percentages)
(i) an oleaginous liquid in a concentration of from 25%-90%,
(ii) a thickening agent in a concentration of from 5% to 35%, and
(iii) a coated particulate material having sun blocking properties in a concentration of from 0.5% to 25%;
b. mixing the oleaginous liquid and thickening agent and heating to a temperature of greater than 80° C.;
c. providing a portion of the oleaginous liquid at a temperature less than 8° C.;
d. mixing the components until the product is sufficiently cooled for packaging.

2. The method of claim 1, further comprising selecting the oleaginous liquid from the group consisting essentially of liquid hydrocarbons, fatty alcohols, vegetable oils, mineral oil, white petrolatum, lanolin alcohol, lanolin, oleyl myristate, methyl, isopropyl and butyl esters of fatty acids and acetoglyceride ester.

3. The method of claim 1, wherein the oleaginous liquid comprises isopropyl myristate.

4. The method of claim 1, further comprising selecting the thickening agent from the group consisting essentiall of polyethylene, polypropylene, polybutylene, polymethylbutylene copolymer, copolymers of olefins and acetates, sorbitan fatty esters, and aliphatic monomers.

5. The method of claim 1, wherein the thickening agent comprises glycerol monostearate.

6. The method of claim 1, further comprising selecting the particulate material from the group consisting essentially of inorganic and organic sunscreen materials.

7. The method of claim 6, further comprising selecting the inorganic sunscreen material from the group consisting essentially of zinc oxide and titanium dioxide.

8. The method of claim 6, wherein the particulate material is coated with dimethicone.

9. The method of claim 1, further comprising adding silicone to the product produced thereby.

10. A method of preparing a sunscreen composition comprising the steps of:
a. forming a first mixture by mixing a thickening agent and an oleaginous liquid to a temperature greater than 100° C.;
b. rapidly mixing the first mixture;
c. heating a second mixture of oleaginous liquid and a sunscreen material to a temperature greater than 100° C.;
d. combining the first and second mixtures;
e. cooling a portion of oleaginous liquid to a temperature less than 8° C. and added to the combined first and second mixtures.

11. A sunscreen material comprising:
f. an oleaginous liquid base in an amount of from 25-90% by weight;
g. a thickening agent in an amount of from 5-35% by weight;
h. a coated particulate sunscreen material in an amount of from 0.5% to 25% by weight.

12. The material of claim 11, wherein said oleaginous liquid base comprises isopropyl myristate.

13. The material of claim 11, wherein said thickening agent comprises glycerol monosterate.

14. The material of claim 11, wherein said coated particulate sunscreen material comprises zinc oxide.

15. The material of claim 14, wherein said zinc oxide is coated with dimethicone.

16. The material of claim 11, wherein said coated particulate sunscreen material comprises titanium dioxide.

17. The material of claim 11, wherein said coated particulate sunscreen material is selected from group consisting essentially of zinc oxide, titanium dioxide, iron oxide, octyl methoxycinnamate, digalloyl trioleate, dioxybenzone, ethyl-4-[bis(hydroxypropyl)] aminobenzoate, octyl salicylate, glyceryl aminobenzoate, homosalate, menthyl anthranilate, octocyrlene, oxybenzone, phenylbenzimidazole sulfonic acid, red petrolatum, sulisobenzone and trolamine salicylate, and combinations thereof.

18. The material of claim 11, wherein said thickening agent is selected from the group consisting essentially of polyethylene, polypropylene, polylene, polymethylbutylene copolymer, copolymers of olefins, sorbitan fatty esters, aliphatic monomers, and combinations thereof.

19. The material of claim 11, wherein said oleaginous liquid base is selected from the group consisting essentially of liquid hydrocarbons, fatty alcohols, vegetable oil, mineral oil, white petrolatum, lanolin alcohol, lanolin, oleyl myristate, methyl/isopropyl/butyl esters of fatty acids, acetoglyceride esters, and combinations thereof.

20. The material of claim 11, wherein silicon is added to the final product.
